(19) United States
(12) Patent Application Publication
(10) Pub. No.: US 2019/0224040 A1
(43) Pub. Date: Jul. 25, 2019

(54) Partial Coverage Condom

(71) Applicant: Blake Peterson, Belleville, MI (US)

(72) Inventor: Blake Peterson, Belleville, MI (US)

(21) Appl. No.: 15/879,972

(22) Filed: Jan. 25, 2018

(51) Int. Cl. A61F 6/04

(52) U.S. Cl. CPC A61F 6/04

(57) ABSTRACT

A partial coverage condom consists of a shaft portion and a head portion, with a head seal connected between the shaft portion and the head portion, just behind the head of the penis. A plurality of openings traverses laterally into the shaft portion, providing the user with enhanced sensation during intercourse.

### FIELD OF THE INVENTION

The present invention relates generally to prophylactic devices. More particularly, the present invention relates to sexual barrier devices.

### BACKGROUND OF THE INVENTION

A condom's purpose is to prevent pregnancy and prevent the spread of sexually transmitted diseases. Pregnancy and diseases which are transferred through fluid are prevented by stopping the transfer of and collecting the fluid during sexual intercourse. Furthermore, a condom lessens the chance of diseases which are transferred by skin to skin contact by covering the shaft of the penis.

There are many condom designs that exist on the market today. A typical condom contains a ring or seal at the base of the penis, and will enclose the penis in its entirety using a latex or latex like material. The shaft of the condom can be made to be tight fitting or loose, and can incorporate features such as ribs or dimples to enhance the feeling for both the male and the female during intercourse.

The problem with existing condom designs is loss of feeling or stimulation for the male participant. Even with the invention of ultra-thin condoms, the complete covering of the penis shaft still degrades the experience of intercourse for the male participant. It will be apparent to those skilled in the art from this disclosure that there exists a need for an improved condom design. This invention addresses the loss of feeling for the male while still achieving the basic function of preventing pregnancy and many diseases.

### BRIEF DESCRIPTION OF THE DRAWINGS

FIG. 1 is a front view of one embodiment of the present invention.

FIG. 2 is a side view of one embodiment of the present invention.

FIG. 3 is a perspective view of one embodiment of the present invention.

FIG. 4 is a perspective view of an alternate embodiment of the present invention.

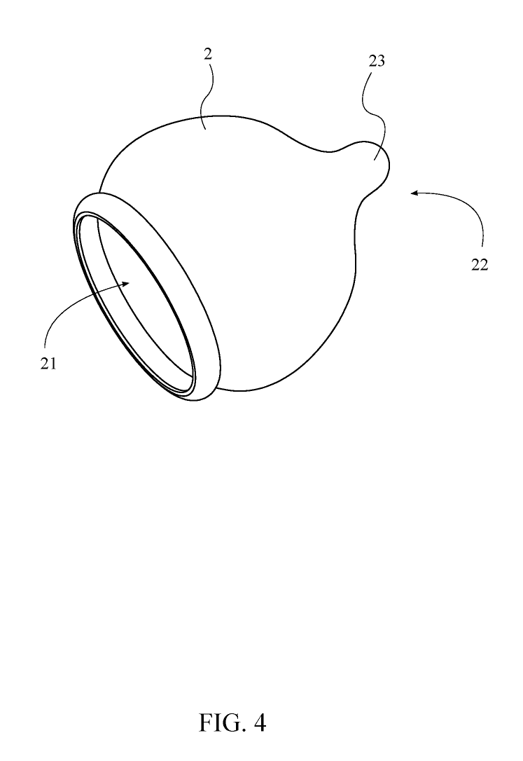

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

The present invention is a male prophylactic contraceptive device intended to prevent pregnancy and disease transmission while providing enhanced sensation for the male participant during intercourse over typical current male contraceptive devices known as condoms. More particularly, the present invention is a modified type of condom device with a plurality of openings allowing a more natural feel during sexual activity with a seal behind the head of the penis for preventing unwanted egress of semen or other fluids from the condom.

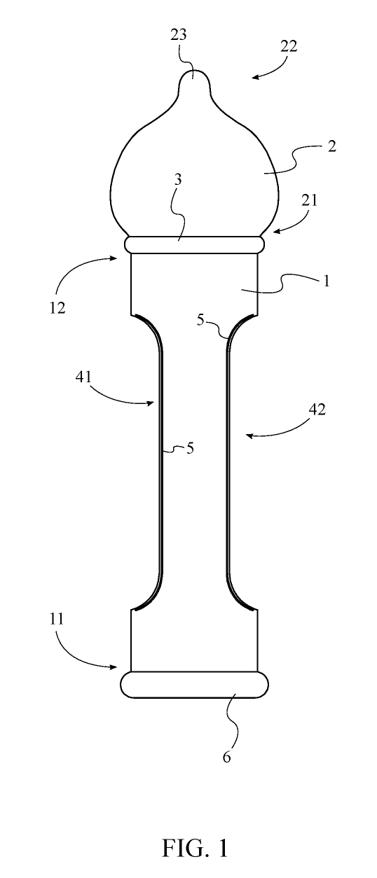

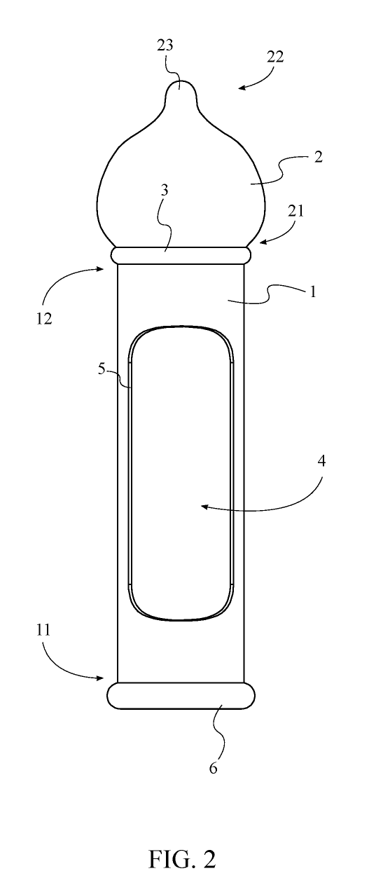

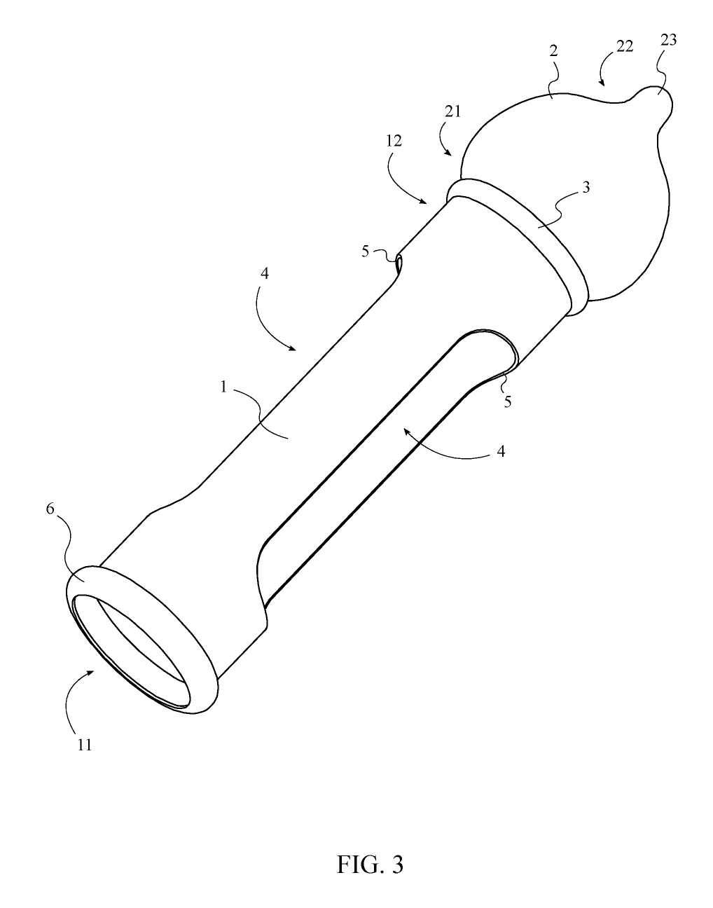

Generally, as illustrated in FIGS. 1-3, the present invention comprises a shaft portion 1, a head portion 2, a head seal 3, and a plurality of openings 4. In the preferred embodiment of the present invention, the shaft portion 1, the head portion 2, and the head seal 3 are made of an elastic material such as, but not limited to, latex, silicone, polyurethane, lambskin, polyisoprene, or other relevant materials. The shaft portion 1 is the main body of the present invention, intended to extend along the majority of the shaft of the penis while in use, extending longitudinally between a proximal end 11 and a distal end 12 of the shaft portion 1. Though it is acknowledged that due to the flexible nature of the material, it may be understood that the shaft portion 1 is preferably constructed to have a generally circular cross section. Thus, the shaft portion 1 may be tubular in various embodiments of the present invention. The longitudinal direction may be understood herein to be defined as parallel to a central axis of the shaft portion 1, wherein the shaft portion 1 is generally tubular or cylindrical. Correspondingly, a lateral direction may be understood herein to be defined any direction perpendicular to the longitudinal direction.

The head portion 2 encloses the head of the penis while in use. The head seal 3 serves to enclose the head of the penis in order to prevent transfer of semen or other fluids discharged from the penis or coming into contact with the penis in order to prevent pregnancy and sexually transmitted diseases. In various embodiments, the head seal 3 may take various forms or means in order to accomplish the aforementioned purpose. In the preferred embodiment of the present invention, the head seal 3 is a band of latex, or other similar material as previously discussed, with diameter less than that of the shaft portion 1 in order to constrict against the penis shaft and prevent fluid passage. In other embodiments, the diameter of the head seal 3 may be equal or similar to that of the shaft portion 1, or may otherwise vary. The head seal 3 may furthermore have a specified radial thickness for increased strength. Furthermore, in the preferred embodiment, a shaft seal 6 is connected to the distal end 12. The shaft seal 6 may take the same form as the head seal 3, or the shaft seal 6 and the head seal 3 may differ in geometry or material. Furthermore, the shaft seal 6 is preferably positioned concentrically with the shaft portion 1.

Furthermore, the head portion 2 comprises an open end 21 and a closed end 22, with the open end 21 being positioned adjacent to the head seal 3 and the closed end 22 being positioned opposite the open end 21 along the head portion 2. It will be apparent that the interiors of the shaft portion 1, the head seal 3, and the head portion 2 are open to each other, providing a passage for the penis of the user to be inserted through the present invention through the proximal end 11 of the shaft portion 1, past the head seal 3, and into the head portion 2. Furthermore, in the preferred embodiment of the present invention, the head portion 2 comprises a fluid reservoir 23 positioned at the closed end 22, opposite the head seal 3, at the top of the penis. The fluid reservoir 23 protrudes longitudinally from the head portion 2, providing a space for fluid ejected from the penis to be collected internal to the head portion 2 prior to removal of the present invention. The fluid reservoir 23 is positioned adjacent to the closed end 22 of the head portion 2, opposite the open end 21.

The distal end 12 of the shaft portion 1 is connected adjacent to the head seal 3, and the head portion 2 is connected adjacent to the head seal 3 opposite the shaft portion 1. Again, notwithstanding the flexible nature of the materials comprising the present invention resulting in inconsistent geometry, the shaft portion 1, the head seal 3, and the head portion 2 may be understood to be concentrically connected to each other. More particularly, the head seal 3 is concentrically connected between the shaft portion 1 and the head portion 2.

The plurality of openings 4 traverses laterally through the shaft portion 1 between the proximal end 11 and the distal end 12 of the shaft portion 1. Each of the plurality of openings 4 is a region of space along the shaft portion 1 not occupied by any physical material. The plurality of openings 4 may be formed during manufacture through removing portions of existing material or through initially forming the shaft portion 1 without any material in the locations desired for the plurality of openings 4. The shape, number, and location of the plurality of openings 4 may vary.

In some embodiments, the plurality of openings 4 comprises a first opening 41 and a second opening 42 traversing longitudinally along the shaft portion 1 between the proximal end 11 and the distal end 12 and positioned laterally opposite each other on the shaft portion 1. It may be noted that in order to accomplish the intended purpose of the present invention, it is ideal for the space occupied by the plurality of openings 4 to approach as close as is feasible to the entire surface area of the shaft portion 1. In some embodiments, the plurality of openings 4 may comprise three or more openings traversing longitudinally between the proximal end 11 and the distal end 12, distributed radially around the shaft portion 1. In some embodiments, the plurality of openings 4 may comprise a geometric pattern or grid of many smaller openings distributed radially around and/or longitudinally along the shaft portion 1 between the proximal end 11 and the distal end 12. In some embodiments, the plurality of openings 4 comprises a plurality of circular openings. Some embodiments of the present invention may further comprise a plurality of reinforcing portions 5 in order to prevent breakage by recuperating any structural durability of the shaft portion 1 diminished by the removal or absence of material due to the presence of the plurality of openings 4. As such, each of the plurality of reinforcing portions 5 may be perimetrically connected around one of the plurality of openings 4. In some embodiments, the plurality of reinforcing portions 5 may be located elsewhere on the shaft portion 1, alternatively or in addition to around the plurality of openings 4. Each of the plurality of reinforcing portions 5 may be, but is not limited to, a region of material with a thickness greater than the general thickness of the shaft portion 1.

In an alternative embodiment of the present invention, the shaft portion 1 and the shaft seal 6 are not included, leaving only the head portion 2 and the head seal 3. This embodiment allows the maximum amount of sensation for the user while still preventing pregnancy and disease due to any fluids or bacterial ejected from the penis or coming into contact with the penis.

Although the invention has been explained in relation to its preferred embodiment, it is to be understood that many other possible modifications and variations can be made without departing from the spirit and scope of the invention as hereinafter claimed.

What is claimed is:

1. A partial coverage condom comprises:
a shaft portion;
a head portion;
a head seal;
a plurality of openings;
the shaft portion extending longitudinally between a proximal end and a distal end of the shaft portion;
the distal end of the shaft portion being connected adjacent to the head seal;
the head portion being connected adjacent to the head seal opposite the shaft portion; and
the plurality of openings traversing through the shaft portion.

2. The partial coverage condom as claimed in claim 1 comprises:
the shaft portion being tubular.

3. The partial coverage condom as claimed in claim 1 comprises:
the shaft portion being made of an elastic material.

4. The partial coverage condom as claimed in claim 1 comprises:
the plurality of openings traversing laterally through the shaft portion between the proximal end and the distal end.

5. The partial coverage condom as claimed in claim 1 comprises:
the head seal being connected between the shaft portion and the head portion; and
the head seal being positioned concentrically with the shaft portion and the head portion.

6. The partial coverage condom as claimed in claim 1 comprises:
the head portion comprises an open end and a closed end;
the open end being positioned adjacent to the head seal; and
the closed end being positioned opposite the open end along the head portion.

7. The partial coverage condom as claimed in claim 1 comprises:
the head portion comprises a fluid reservoir; and
the fluid reservoir being positioned opposite the head seal.

8. The partial coverage condom as claimed in claim 1 comprises:
the fluid reservoir being positioned adjacent to a closed end of the head portion opposite an open end of the head portion.

9. The partial coverage condom as claimed in claim 1 comprises:
the plurality of openings comprises a first opening and a second opening;
the first opening and the second opening traversing longitudinally along the shaft portion between the proximal end and the distal end; and
the first opening and the second opening being positioned laterally opposite each other on the shaft portion.

10. The partial coverage condom as claimed in claim 1 comprises:
a plurality of reinforcing portions; and
each of the plurality of reinforcing portions being perimetrically connected around one of the plurality of openings.

11. The partial coverage condom as claimed in claim 1 comprises:
a shaft seal;
the shaft seal being connected to the distal end; and
the shaft seal being positioned concentrically with the shaft portion.

12. A partial coverage condom comprises:
a shaft portion;
a head portion;
a head seal;
a plurality of openings;
the shaft portion extending longitudinally between a proximal end and a distal end of the shaft portion;
the distal end of the shaft portion being connected adjacent to the head seal;
the head portion being connected adjacent to the head seal opposite the shaft portion;
the plurality of openings traversing laterally through the shaft portion between the proximal end and the distal end;
the shaft portion being tubular; and
the shaft portion being made of an elastic material.

13. The partial coverage condom as claimed in claim 12 comprises:
the head seal being connected between the shaft portion and the head portion; and
the head seal being positioned concentrically with the shaft portion and the head portion.

14. The partial coverage condom as claimed in claim 12 comprises:
the head portion comprises an open end, a closed end, and a fluid reservoir;
the open end being positioned adjacent to the head seal;
the closed end being positioned opposite the open end along the head portion; and
the fluid reservoir being positioned opposite the head seal, adjacent to the closed end.

15. The partial coverage condom as claimed in claim 12 comprises:
the plurality of openings comprises a first opening and a second opening;
the first opening and the second opening traversing longitudinally along the shaft portion between the proximal end and the distal end; and
the first opening and the second opening being positioned laterally opposite each other on the shaft portion.

16. The partial coverage condom as claimed in claim 12 comprises:
a plurality of reinforcing portions; and
each of the plurality of reinforcing portions being perimetrically connected around one of the plurality of openings.

17. The partial coverage condom as claimed in claim 12 comprises:
a shaft seal;
the shaft seal being connected to the distal end; and
the shaft seal being positioned concentrically with the shaft portion.

18. A partial coverage condom comprises:
a shaft portion;
a head portion;
a head seal;
a plurality of openings;
the shaft portion extending longitudinally between a proximal end and a distal end of the shaft portion;
the distal end of the shaft portion being connected adjacent to the head seal;
the head portion being connected adjacent to the head seal opposite the shaft portion;
the plurality of openings traversing laterally through the shaft portion between the proximal end and the distal end;
the shaft portion being tubular;
the shaft portion being made of an elastic material;
the head seal being connected between the shaft portion and the head portion;
the head seal being positioned concentrically with the shaft portion and the head portion;
the head portion comprises an open end, a closed end, and a fluid reservoir;
the open end being positioned adjacent to the head seal;
the closed end being positioned opposite the open end along the head portion;
the fluid reservoir being positioned opposite the head seal, adjacent to the closed end;
a shaft seal;
the shaft seal being connected to the distal end; and
the shaft seal being positioned concentrically with the shaft portion.

19. The partial coverage condom as claimed in claim 18 comprises:
the plurality of openings comprises a first opening and a second opening;
the first opening and the second opening traversing longitudinally along the shaft portion between the proximal end and the distal end; and
the first opening and the second opening being positioned laterally opposite each other on the shaft portion.

20. The partial coverage condom as claimed in claim 18 comprises:
a plurality of reinforcing portions; and
each of the plurality of reinforcing portions being perimetrically connected around one of the plurality of openings.
